SEQUENCE LISTING (24 sequences)

SEQ ID NO: 1 - protein, 684 aa, Caenorhabditis elegans
  misc_feature: Long form of the SES-1 amino acid sequence
MPPRKRKLEELKNEQGEQGSYKCHLCGQCFYRGCGLASHLRRHAPVTFDCEHCAYTCKHKYAYDRHLLQSHPELVESGPLVRMFKNEDEDAPMPILEREADVSDDAVKYEALSSFSEPEPISYKCPLCISTFGSHARAVYHILSHRVKLYQAPKSLKFFSRKTMQALGGFRLESQLMIKWRLQYDNRSVDEKRTRLWRYMEENFGHEHLKQPKLESDNPSTSSSFESQTNLNDSSIIKKKLVMKCGTVFGQRLIHDNTQYYLCRNCPYVSWNVSSLWRHFRHHIQKSKQSWTCIACSYSSSSRVKIDLHVKMHKEMPEIDLEFATWLRYERRINKNDLNKPTNKKKKPDGGNGSNHSDMRSLHAFLSLKNSKNNVVKHDIDAPTLHPLSPAPKLVAMTQFDFGEIVTYKSVNPLHQINKNNSNPTVLPNKRNSIKTSKSDTQIALSVKQSSSMKMVKVSPGKVYQLPKTSKFYRPESPDSLASNNSAHGDEIESTSSDQFQQSVKVPKYEDFLNMKPVMPYFQKQRHPLEAIAMYEKAKREYEKNHCFPNLPIFEFNIEYKNLHPLAKAQYGKNNMKEYFLNEMEVEKSRECTDCPFKHNDLQQFRLHRDKHFYGGSHTCPECNYSSNNHNQVVEHTFVDHYLSDVRLVEGLPSSDSEDDNIPVPPDTPQRKKKAPKRGKRRGW

SEQ ID NO: 2 - protein, 623 aa, Caenorhabditis elegans
  misc_feature: Short form of the SES-1 amino acid sequence
MPPRKRKLEELKNEQGEQGSYKCHLCGQCFYRGCGLASHLRRHAPVTFDCEHCAYTCKHKYAYDRHLLQSHPELVESGPLVRMFKNEDEDAPMPILEREADVSDDAVKYEALSSFSEPEPISYKCPLCISTFGSHARAVYHILSHRVKLYQAPKSLKFFSRKTMQALGGFRLESQLMIKWRLQYDNRSVDEKRTRLWRYMEENFGHEHLKQPKLESDNPSTSSSFESQTNLNDSSIIKKKLVMKCGTVFGQRLIHDNTQYYLCRNCPYVSWNVSSLWRHFRHHIQKSKQSWTCIACSYSSSSRVKIDLHVKMHKEMPEIDLEFATWLRYERRINKNDLNKPTNKKKKPDGGNGSNHSDMRSLHAFLSLKNSKNNVVKHDIDAPTLHPLSPAPKLVAMTQFDFGEIVTYKSVNPLHQINKNNSNPTVLPNKRNSIKTNQFQQSVKVPKYEDFLNMKPVMPYFQKQRHPLEAIAMYEKAKREYEKNHCFPNLPIFEFNIEYKNLHPLAKAQYGKNNMKEYFLNEMEVEKSRECTDCPFKHNDLQQFRLHRDKHFYGGSHTCPECNYSSNNHNQVVEHTFVDHYLSDVRLVEGLPSSDSEDDNIPVPPDTPQRKKKAPKRGKRRGW

SEQ ID NO: 3 - DNA, 2055 bp, Caenorhabditis elegans
  misc_feature: Long form of ses-1 cDNA from ATG to stop
atgccacctcgcaagcgcaaacttgaggagttgaagaatgaacaaggagagcaggggtcatacaaatgccatctttgtggacagtgcttctaccgtggatgtggattagcaagtcacctgcgacgacacgcaccagtcacatttgactgtgagcattgtgcttacacgtgtaagcacaaatatgcgtatgatcgtcatctgttgcaatcgcatcctgagctcgtggaatctggcccactagttcgaatgtttaagaatgaagatgaagatgctccaatgccgattctcgagcgagaagcagatgtatccgatgatgccgtcaagtatgaggcactctcttctttctcggaacctgaaccaatcagctacaaatgtccattgtgtatatccacgtttggcagtcacgctagagcagtctaccatattttgtcacaccgtgtcaagttgtatcaagcaccgaaaagcctgaaattcttctcgaggaaaacaatgcaagctcttggaggatttcgtttagagtcgcaactgatgatcaaatggagattacaatacgataatcgaagtgtggacgagaagcgaacacggttatggcggtatatggaagaaaattttggacatgaacatctgaagcaaccaaagctggaaagtgataatccttcgacaagtagctcttttgaatcacaaacaaacttgaacgattcttcgataatcaaaaagaaacttgtgatgaagtgtggtactgttttcggtcaaagactcattcacgacaacacccaatattatttgtgccgcaattgcccatacgtttcgtggaatgtttcttcactgtggcgacatttccgtcatcatattcaaaaatcgaaacaatcttggacgtgtatcgcatgctcatacagctcatcgagccgggtgaaaattgatctacatgtgaaaatgcataaggaaatgccggaaattgatttggagtttgcaacttggcttcgctatgaacgaagaatcaacaagaacgacttgaataaaccgactaacaaaaaaaagaaacccgatggaggaaacggttcaaatcacagtgatatgagaagtcttcacgcatttcttagtcttaaaaattctaagaacaatgttgtcaaacacgatatcgatgctccaactttgcacccattatccccagcgcccaagctggtcgcgatgactcaatttgacttcggagaaattgtcacttacaaaagcgtcaatccgctgcaccaaatcaacaagaacaatagcaatccgacggtcctgccgaacaagcgaaatagcattaaaaccagtaaaagtgatactcaaatcgcgctcagcgttaagcagtctagtagtatgaagatggtgaaagtttctcctggaaaagtttatcagcttcctaagacgtccaaattttaccgacctgaatcacctgactcattagcatcaaataattctgctcacggagacgagattgagtcaacgtcttcagatcaattccaacaatccgtgaaagttccaaagtacgaagactttttgaacatgaagccagttatgccatatttccaaaaacaacgacatccactggaagcgattgctatgtacgaaaaggcaaaaagggaatacgaaaagaatcactgtttcccgaatcttccaattttcgaattcaacattgagtacaagaacttgcatccactcgccaaggcacagtatgggaaaaacaatatgaaagagtattttttgaatgaaatggaagtagagaaatctcgcgaatgcactgactgcccattcaaacacaacgatctgcagcagttccgtctccaccgtgacaagcatttctacggtggatcacacacctgtccggagtgcaactattcatcgaacaaccataatcaagtggtcgagcacacgtttgtggatcactatttgagcgatgtgagattggttgaagggcttccgtcttcggattcggaggacgataatattccggtacctcctgatactccgcagcggaagaaaaaagcgccgaagcgtggcaaacgtcgtggctggtaa

SEQ ID NO: 4 - DNA, 2497 bp, Caenorhabditis elegans
  misc_feature: Long form of ses-1 cDNA from ATG to stop and noncoding 3′ end region
atgccacctcgcaagcgcaaacttgaggagttgaagaatgaacaaggagagcaggggtcatacaaatgccatctttgtggacagtgcttctaccgtggatgtggattagcaagtcacctgcgacgacacgcaccagtcacatttgactgtgagcattgtgcttacacgtgtaagcacaaatatgcgtatgatcgtcatctgttgcaatcgcatcctgagctcgtggaatctggcccactagttcgaatgtttaagaatgaagatgaagatgctccaatgccgattctcgagcgagaagcagatgtatccgatgatgccgtcaagtatgaggcactctcttctttctcggaacctgaaccaatcagctacaaatgtccattgtgtatatccacgtttggcagtcacgctagagcagtctaccatattttgtcacaccgtgtcaagttgtatcaagcaccgaaaagcctgaaattcttctcgaggaaaacaatgcaagctcttggaggatttcgtttagagtcgcaactgatgatcaaatggagattacaatacgataatcgaagtgtggacgagaagcgaacacggttatggcggtatatggaagaaaattttggacatgaacatctgaagcaaccaaagctggaaagtgataatccttcgacaagtagctcttttgaatcacaaacaaacttgaacgattcttcgataatcaaaaagaaacttgtgatgaagtgtggtactgttttcggtcaaagactcattcacgacaacacccaatattatttgtgccgcaattgcccatacgtttcgtggaatgtttcttcactgtggcgacatttccgtcatcatattcaaaaatcgaaacaatcttggacgtgtatcgcatgctcatacagctcatcgagccgggtgaaaattgatctacatgtgaaaatgcataaggaaatgccggaaattgatttggagtttgcaacttggcttcgctatgaacgaagaatcaacaagaacgacttgaataaaccgactaacaaaaaaaagaaacccgatggaggaaacggttcaaatcacagtgatatgagaagtcttcacgcatttcttagtcttaaaaattctaagaacaatgttgtcaaacacgatatcgatgctccaactttgcacccattatccccagcgcccaagctggtcgcgatgactcaatttgacttcggagaaattgtcacttacaaaagcgtcaatccgctgcaccaaatcaacaagaacaatagcaatccgacggtcctgccgaacaagcgaaatagcattaaaaccagtaaaagtgatactcaaatcgcgctcagcgttaagcagtctagtagtatgaagatggtgaaagtttctcctggaaaagtttatcagcttcctaagacgtccaaattttaccgacctgaatcacctgactcattagcatcaaataattctgctcacggagacgagattgagtcaacgtcttcagatcaattccaacaatccgtgaaagttccaaagtacgaagactttttgaacatgaagccagttatgccatatttccaaaaacaacgacatccactggaagcgattgctatgtacgaaaaggcaaaaagggaatacgaaaagaatcactgtttcccgaatcttccaattttcgaattcaacattgagtacaagaacttgcatccactcgccaaggcacagtatgggaaaaacaatatgaaagagtattttttgaatgaaatggaagtagagaaatctcgcgaatgcactgactgcccattcaaacacaacgatctgcagcagttccgtctccaccgtgacaagcatttctacggtggatcacacacctgtccggagtgcaactattcatcgaacaaccataatcaagtggtcgagcacacgtttgtggatcactatttgagcgatgtgagattggttgaagggcttccgtcttcggattcggaggacgataatattccggtacctcctgatactccgcagcggaagaaaaaagcgccgaagcgtggcaaacgtcgtggctggtaaagattatattttattaaattttatcaagtttttttaaaggtaaatgtttaaatttattttaaatttaaatttatttttgtttctttttttttcaattaattgtttttaagtttttcaatgttaggtttgtcgtttaatttttttcaaatttctaataatattcccttcccttcccatccccgctcaaatagctgcctcatagtttagtaaatttctaaataatattatttcaataccatcacatattgtcagcgctttctgtcatttttttaattttcattcatcctggtacctcacttttggcacgtgtccaacaaccaccctgacatcaacccacccggactttctcgactaggtttttttgaaattcttcccccattttattttctttgaccattttatgtttgtgttttgaatgaaatgtgttttcgaatttttaaaa

SEQ ID NO: 5 - DNA, 1872 bp, Caenorhabditis elegans
  misc_feature: Short form of the ses-1 cDNA from ATG to stop (yk247e5)
atgccacctcgcaagcgcaaacttgaggagttgaagaatgaacaaggagagcaggggtcatacaaatgccatctttgtggacagtgcttctaccgtggatgtggattagcaagtcacctgcgacgacacgcaccagtcacatttgactgtgagcattgtgcttacacgtgtaagcacaaatatgcgtatgatcgtcatctgttgcaatcgcatcctgagctcgtggaatctggcccactagttcgaatgtttaagaatgaagatgaagatgctccaatgccgattctcgagcgagaagcagatgtatccgatgatgccgtcaagtatgaggcactctcttctttctcggaacctgaaccaatcagctacaaatgtccattgtgtatatccacgtttggcagtcacgctagagcagtctaccatattttgtcacaccgtgtcaagttgtatcaagcaccgaaaagcctgaaattcttctcgaggaaaacaatgcaagctcttggaggatttcgtttagagtcgcaactgatgatcaaatggagattacaatacgataatcgaagtgtggacgagaagcgaacacggttatggcggtatatggaagaaaattttggacatgaacatctgaagcaaccaaagctggaaagtgataatccttcgacaagtagctcttttgaatcacaaacaaacttgaacgattcttcgataatcaaaaagaaacttgtgatgaagtgtggtactgttttcggtcaaagactcattcacgacaacacccaatattatttgtgccgcaattgcccatacgtttcgtggaatgtttcttcactgtggcgacatttccgtcatcatattcaaaaatcgaaacaatcttggacgtgtatcgcatgctcatacagctcatcgagccgggtgaaaattgatctacatgtgaaaatgcataaggaaatgccggaaattgatttggagtttgcaacttggcttcgctatgaacgaagaatcaacaagaacgacttgaataaaccgactaacaaaaaaaagaaacccgatggaggaaacggttcaaatcacagtgatatgagaagtcttcacgcatttcttagtcttaaaaattctaagaacaatgttgtcaaacacgatatcgatgctccaactttgcacccattatccccagcgcccaagctggtcgcgatgactcaatttgacttcggagaaattgtcacttacaaaagcgtcaatccgctgcaccaaatcaacaagaacaatagcaatccgacggtcctgccgaacaagcgaaatagcattaaaaccaatcaattccaacaatccgtgaaagttccaaagtacgaagactttttgaacatgaagccagttatgccatatttccaaaaacaacgacatccactggaagcgattgctatgtacgaaaaggcaaaaagggaatacgaaaagaatcactgtttcccgaatcttccaattttcgaattcaacattgagtacaagaacttgcatccactcgccaaggcacagtatgggaaaaacaatatgaaagagtattttttgaatgaaatggaagtagagaaatctcgcgaatgcactgactgcccattcaaacacaacgatctgcagcagttccgtctccaccgtgacaagcatttctacggtggatcacacacctgtccggagtgcaactattcatcgaacaaccataatcaagtggtcgagcacacgtttgtggatcactatttgagcgatgtgagattggttgaagggcttccgtcttcggattcggaggacgataatattccggtacctcctgatactccgcagcggaagaaaaaagcgccgaagcgtggcaaacgtcgtggctggtaa

SEQ ID NO: 6 - DNA, 2314 bp, Caenorhabditis elegans
  misc_feature: Short form of ses-1 cDNA from ATG to stop and noncoding 3′ end region
atgccacctcgcaagcgcaaacttgaggagttgaagaatgaacaaggagagcaggggtcatacaaatgccatctttgtggacagtgcttctaccgtggatgtggattagcaagtcacctgcgacgacacgcaccagtcacatttgactgtgagcattgtgcttacacgtgtaagcacaaatatgcgtatgatcgtcatctgttgcaatcgcatcctgagctcgtggaatctggcccactagttcgaatgtttaagaatgaagatgaagatgctccaatgccgattctcgagcgagaagcagatgtatccgatgatgccgtcaagtatgaggcactctcttctttctcggaacctgaaccaatcagctacaaatgtccattgtgtatatccacgtttggcagtcacgctagagcagtctaccatattttgtcacaccgtgtcaagttgtatcaagcaccgaaaagcctgaaattcttctcgaggaaaacaatgcaagctcttggaggatttcgtttagagtcgcaactgatgatcaaatggagattacaatacgataatcgaagtgtggacgagaagcgaacacggttatggcggtatatggaagaaaattttggacatgaacatctgaagcaaccaaagctggaaagtgataatccttcgacaagtagctcttttgaatcacaaacaaacttgaacgattcttcgataatcaaaaagaaacttgtgatgaagtgtggtactgttttcggtcaaagactcattcacgacaacacccaatattatttgtgccgcaattgcccatacgtttcgtggaatgtttcttcactgtggcgacatttccgtcatcatattcaaaaatcgaaacaatcttggacgtgtatcgcatgctcatacagctcatcgagccgggtgaaaattgatctacatgtgaaaatgcataaggaaatgccggaaattgatttggagtttgcaacttggcttcgctatgaacgaagaatcaacaagaacgacttgaataaaccgactaacaaaaaaaagaaacccgatggaggaaacggttcaaatcacagtgatatgagaagtcttcacgcatttcttagtcttaaaaattctaagaacaatgttgtcaaacacgatatcgatgctccaactttgcacccattatccccagcgcccaagctggtcgcgatgactcaatttgacttcggagaaattgtcacttacaaaagcgtcaatccgctgcaccaaatcaacaagaacaatagcaatccgacggtcctgccgaacaagcgaaatagcattaaaaccaatcaattccaacaatccgtgaaagttccaaagtacgaagactttttgaacatgaagccagttatgccatatttccaaaaacaacgacatccactggaagcgattgctatgtacgaaaaggcaaaaagggaatacgaaaagaatcactgtttcccgaatcttccaattttcgaattcaacattgagtacaagaacttgcatccactcgccaaggcacagtatgggaaaaacaatatgaaagagtattttttgaatgaaatggaagtagagaaatctcgcgaatgcactgactgcccattcaaacacaacgatctgcagcagttccgtctccaccgtgacaagcatttctacggtggatcacacacctgtccggagtgcaactattcatcgaacaaccataatcaagtggtcgagcacacgtttgtggatcactatttgagcgatgtgagattggttgaagggcttccgtcttcggattcggaggacgataatattccggtacctcctgatactccgcagcggaagaaaaaagcgccgaagcgtggcaaacgtcgtggctggtaaagattatattttattaaattttatcaagtttttttaaaggtaaatgtttaaatttattttaaatttaaatttatttttgtttctttttttttcaattaattgtttttaagtttttcaatgttaggtttgtcgtttaatttttttcaaatttctaataatattcccttcccttcccatccccgctcaaatagctgcctcatagtttagtaaatttctaaataatattatttcaataccatcacatattgtcagcgctttctgtcatttttttaattttcattcatcctggtacctcacttttggcacgtgtccaacaaccaccctgacatcaacccacccggactttctcgactaggtttttttgaaattcttcccccattttattttctttgaccattttatgtttgtgttttgaatgaaatgtgttttcgaatttttaaaa

SEQ ID NO: 7 - DNA, 9260 bp, Caenorhabditis elegans
  misc_feature: sequence of yBy1015 that complements the defect in the ses-1 null mutant
gatccagcaaaacttgtcaactctgacttgtctgtttccatcaaagaaggttccaattaccggttaagctttgctttccatgtgcaacgcgaaattacgtccggattgcagttagtttttttgtgtgtttaatcttaacttaaaataataaattgtttcagctacaaacataaggtgaaacgatccggtatcactgtggagaatgagaagtacatgatgggaagctacgcgccaaaacttgagattcaggagtacaaatcaccaaacgaagaagctccgtctggaatgatgcatcgtggaaagtacaaggtgtactcgaaaattaccgacgatgacaacaatgtgtatcttgactggcaatggacccttcacattaccaaggagtagaataattttccgcctttactgaacctcatatttatattcaaattctcaatgaaaatgacttttttctccaatttaccgcccaccccaaatgttttccttgttttatggttattattgattttgatgcactagttgtgttgtgtgatgcctctcccctttccccgccaaattgaaatgatatataaagtgatatttcataatcatttaaaacaaagtgtactgttttgtgaaacgttggaaactaaccaaaaaacactaacttgctgcgtttccaaaaacttaagatcctaatacgcatttacacgttgttccgcaatattcacgtggtttcagctgggccggaacattgtattaatttcagaattagagattttattttgatacaataagaattgaactacaaaacgttacaactttgaaaatttaaaagagtactaacggtttggaagttttgaacgtataaacccaaaatgtgttaaaataaattaatttcgtaataaaaagtcaaaaaaatttgaatagacttttctcacaaaaatccaaacttaaaaaaaaatttaccagaaactttgtagcaaattttcagatctttttactgaatacctaagtatttttggtactcgtgtgttacctaaaattactctgtagttctaactttcaaaattactatgtagttctaacttttaaagttactttaagattagtaaattgaattcgtgcaaaatgtttgaaaattgattttcaagacattaaatgatgatcaccattacaaaaatctttgcaaaaacttactgccgtaaattaaaaagaatttatcgaatcacaccactacgtgtgggtgtagtctgaagaaattcatgctttcttgctccattaggaaatacttaatgaaattggaaattttatcggtgctgggccagaagagccaacaagatccattttgaatttcctcaacttggaatttgaatttgataatatgagctcgttgctccacctcatgcttctttttagttttttgaatattgtgacaactagtcgcgttgagtgcatttgagtgcattgtggttctggaatgtgatccactaaccagctgctattccttgaaaggtaagttacaatagtttttctttactttacaaatcgataaagtcaatcattgactcaactttcgatctgagaaatttctttcaaatatttcaaggtgcgctgatttcagctgtcaaattattttataaccaaaagtttttgacatgatctcccataagttttctaacaacctttggatatgttatatcattaaatatattcattaatatcaaagttgaaaattttattggtttatgaaattcactgaaaaatgctatcaaatgtgagcatttatggggttattcaagtaatcttgcttcatcagttatgggacattcgtaaatagtctcgctattggtcttgcacgcatacggatcatttcaaaattagtcttgaatgcaagcctaacagaggaaatacgttattcatttttcaggaggcttcccatttgtttatggtaacaaaaaggaagaagactggcgtgcaggaggaaaacgatacagtatgtttttggcaagaaacgtggagactgtccgttgtgttttatacgataattccgagttctgaactagacttaacgtcaacagacattctaaaaaactattccttacattgaaaatgaagaggttttctgttctaaatgattttattgtaataaatatttcttatgtttcttaatcttatgtagacatgaaagctgtttagttgttcttttgaaaagaaattaagatgaaatgaaagagaaaggaagcaacgggtttttcgatttttcttcaaaatcagctgttgacatttcttattatatggttaaaattgatatttctattgaaaaaaaacatcaagtttgaaatcattttcgaaaactgaagttcaaatttctacgcctcttgtgttcatcttaacctaaattaaagcttagcttaactcaaataagacttttcaaagtctgaaattattaagtctagatacacctccccctccccctcactgttcttttttactttcaatcgatcaatatctgaacagtatacaactacgctctccactctcattagaaaataacagtgtaccttttcgatgcggtacgcgtcgcggcaccaacgcggcacacacgcagcgcagtcccatctactttttctcggtggttcattgcacaccgatcctcacccagcacacagttttctctccttaacagttggggctgacattgctaactcttccctccgctcacctgtttattctcctttttctagttgtctcgcggtctgcgtcttctcattttatatacatttagagtttagggtgagaacccaatttttcatggtccatttttgttggctaaattctttacatgttgaatattttaaatgctaccacatttggacttactctagaataagttatcttaagacgagttattttagactagactaaccagttaggaatattttatcaatttccgattttgaaacccaaaaaatcaatgttaatttagcactgaaacatgctggtaggcactcaatattttaagcttctagctagaacacttaggactttacttacggaatagccttttagacttttagttagccgtttggggttaggaaaaacaaagaaccctagaagctatgtttccgttgagaaccaatgaccatctatctttcaacgaacatttcacctaataacagagtcatgagattgtgacttgtaaatatttttttccatctttcccctgtttcagtcgtcatttgaacgttctctttcttttcttcctcgacagtgaaaataataaacgtaaaaggttttaaaatcacccattttcaaattccatcgtgtaatttcttatcacaatagtacccacaatcgcttctcgtttcccagcagttatcttaaagcattagccaacatctgttgtccggccgttctcatctcttccgctgtctacgtctctatccttcgtctcactaattcacccttcttttctctctccgtagggctagggtgcgcgcctcgcggccagtagaactgtgttgtagatgcgaaaagagtgcggtctacttctaagcgcagacatattgtcaacgggggctgcggacgacgcgggagccgccagttttggaagtgtagtactcgcattccccccccccccccccccccccacatgcgcttcgttttcccttgacttccattatcactgtcaattctcttgtcaactgccatttgtataaatcatcaatcctatgttaatattatttcagtagaagacgatgccacctcgcaagcgcaaacttgaggagttgaagaatgaacaaggagaggtaggacagcgttgagattttttttcaaaaacttaatgatttaattttagcaggggtcatacaaatgccatctttgtggacagtgcttctaccgtggatgtggattagcaagtcacctgcgacgacacgcaccagtcacatttgactgtgagcattgtgcttacacgtgtaagcacaaatatgcgtatgatcgtcatctgttgcaatcgcatcctgagctcgtggaatctggcccactagttcgaatgtttaagaatgaagatgaagatgctccaatgccgattctcgagcgagaagcagatgtatccgatgatgccgtcaagtatgaggcactctcttctttctcggaacctgaaccaatcagctacaaatgtccattgtgtatatccacgtttggcagtcacgctagagcagtctaccatattttgtcacaccgtgtcaagttgtatcaagcaccgaaaagcctgaaattcttctcgaggaaaacaatgcaagctcttggaggatttcgtttagagtcgcaactgatgatcaaatggagattacaatacgataatcgaagtgtggacgagaagcgaacacggttatggcggtatatggaagaaaattttggacatgaacatctgaagcaaccaaagctggaaagtgataatccttcgacaagtagctcttttgagtgagttttttgcccaacgagcttttgatagaaaaaaataatgttagaaaccatgagtcactttttcatttcaaaattaatatacaatactaaccacatctttcagatcacaaacaaacttgaacgattcttcgataatcaaaaagaaacttgtgatgaagtgtggtactgttttcggtcaaagactcattcacgacaacacccaatattatttgtgccgcaattgcccatacgtttcgtggaatgtttcttcactgtggcgacatttccgtcatcatattcaaaaatcgaaacaatcttggacgtgtatcgcatgctcatacagctcatcgagccgggtgaaaattgatctacatgtgaaaatgcataaggaaatgccggaaattgatttggaggtaagaaatatatcatcataaccaaaattaaataattttatttaaaaaaaaaatttcagtttgcaacttggcttcgctatgaacgaagaatcaacaagaacgacttgaataaaccgactaacaaaaaaaagaaacccgatggaggaaacggttcaaatcacagtgatatgagaagtcttcacgcatttcttagtcttaaaaattctaagaacaatgttgtcaaacacgatatcgatgctccaactttgcacccattatccccagcgcccaagctggtcgcgatgactcaatttgacttcggagaaattgtcacttacaaaagcgtcaatccgctgcaccaaatcaacaagaacaatagcaatccgacggtcctgccgaacaagcgaaatagcattaaaaccagtaaaagtgatactcaaatcgcgctcagcgttaagcagtctagtagtatgaagatggtgaaagtttctcctggaaaagtttatcagcttcctaagacgtccaaattttaccgacctgaatcacctgactcattagcatcaaataattctgctcacggagacgagattgagtcaacgtcttcagatcaattccaacaatccgtgaaagttccaaagtacgaagactttttgaacatgaagccagttatgccatatttccaaaaacaacgacatccactggaagcgattgctatgtacgaaaaggcaaaaagggaatacgaaaagaatcactgtttcccgaatcttccaattttcgaattcaacattgagtacaagaacttgcatccactcgccaaggcacagtatgggaaaaacaatatgaaagagtattttttgaatgaaatggaagtagagagtaagttaacgagtcttcaacgaagtacatattctgaaaaaaactagaacatttttatcaaatggctctttttcagaatctcgcgaatgcactgactgcccattcaaacacaacgatctgcagcagttccgtctccaccgtgacaagcatttctacggtggatcacacacctgtccggagtgcaactattcatcgaacaaccataatcaagtggtcgagcacacgtttgtggatcactatttgagcgatgtgagattggttgaagggcttccgtcttcggattcggaggacgataatattccggtacctcctgatactccgcagcggaagaaaaaagcgccgaagcgtggcaaacgtcgtggctggtaaagattatattttattaaattttatcaagtttttttaaaggtaatgtttaaatttattttaaatttaaatttatttttgtttctttttttttcaattaattgtttttaagtttttcaatgttaggtttgtcgtttaatttttttcaaatttctaataatattcccttcccttcccatccccgctcaaatagctgcctcatagtttagtaaatttctaaataatattatttcaataccatcacatattgtcagcgctttctgtcatttttttaattttcattcatcctggtacctcacttttggcacgtgtccaacaaccaccctgacatcaacccacccggactttctcgactaggtttttttgaaattcttcccccattttattttctttgaccattttatgtttgtgttttgaatgaaatgtgttttcgaatttttaaaatttgttttagactttggaatctgggtaaaaatatattactgaatgtcgtcagttttgatgcttgcacagccaactgcagatacacaaaaggcaaaattgcggacttaggagtgttggatatacggaaatgaacatacgtgttaatcagtttaacggattgcaaattcaatgggttttcaaattttcagatatattatatgtagtatcgaagcaattaaggtaaaagcgatagatataaacattatgaagcctgtatgaaattggcttctattaaatatttacattaatcgaataacattttacaacttgcaacgtatgactcaatcttttataaacgccagaaaaccatttaaaacggaagcaccgattttaacactaatttttcaaacaagccggatcagactaacacggactaaacttagacatagataaagtgttgaaaaagaacctatattgacatgtgagtagctaggactggtctgaatacatcagaacaggaaaaaataacagaatatctcttaccaaaactataaatagaaaaaaaagttgatcaaggctgtagcagttcagagtaaagccaaagtaagatattgataaggagagtttctcagagtaaaaccatgtggattagcaaaaaatgctgaaactatttcgtcgagccattccaattcagaaatcttactgtcagatttttcgacactttttgactgtcgcatatccaagacgttcctgtttttgaattataccgaagattttcggaattcatttggctttttctctgacttttcatcttttatggaaaatctagtaattctggcttcttccaattctttacagtttgtttgaattttctggatacgaagcattttattacacaggtacagaaatggttatctcgaataaatataacactttagtacggatagggaatgaaattgacattattcacaacgattttcagcatcttttgagtagttggctagccagctgttttgatttacaatttacaatttgtcgttttcctcttgctcctttgtttctgaaaaatgtataatttcatatacatataaattcagaaaatttgttgaaaatacaacgtttactcacttttctcactctgattctcaagccacttctccatttcagcagcttcgtgctcggttggcaacttgttgctcacagcggctgcaagtccaacgtcggtttgtggctcagcgtttgccttgtaagcatcctgagaggttgaggcgctgcttgatggctctccgctggcagcagttacctctgaaattagataattcaattttgtttttcttttcgtaacttactgagggctgataattggtcacccaaactttttccgtcacccatatcaattagatcacgagcttcggcggcttgcttttccccgtcgcggtttgacacaaaacgatcatatttttcaaaaacactgttcaatgaatcattgaccataagaagttcatctaaaacacaagattttagtttcagtcgcaaaaaacacccattcttacaagtaacttcatcattgctcacgtagcggatcaggtccagcacgcgttgttgcatgtgacggcaagtgtcgttgaggtccgatagcaattgcagctcgtcggcggtctccttgcgtggcacaacgtcggtgagcgtctcgcggaacactttgatgttttgattgaccacgtcaaggtctgcccgcaatttggtcaattgcgctggtgttgcggagattggctcttgtccctgctcgcggatggtgagaacgtcgtatgtctaaaatgtagtaattgttacgtgttttttgaataaaaaaacaaacaaactttgtaatgaaacttttcaaaatttttcagaaatgtttcatgacggttcaaaattttcaacaaaaaaaaaacgctaagtctagctaaaactccaattttgattacttttccgtaccgcagcagctggaaaacaatttttgttgaattatcatcccttatcgaatttttggtagtaatttttgaacattccgttgactaaggtacttttaaagccgataaggcaatttttggttagatatgactttggatgtattttaatcgacgaaccaacgtaagataaacccactaaatattacaacaagattgcttgaaaaatgctatgatacacctgctgatgtataaaaaaaacaggccaaaaatggatattttagctagaagtagcgttttttttttgaaaattttgaacaatcataaataatttgtgaaacattttttaaaaatctcataacgaaataagctcatttaagcacatcttatgtctatacattgacataaacgcttttcgttagccctcctatacaaaactctattcatatttgttattgactagtgacgctgacgctttcacaactttgaataataataataaagcttgaaatcacaaaacaatatctaccgattgggggttttgttggccttgctgtggttgttgttgctgttgctgattttgttgttgttgatcatctagggttgccggtggaggttgattaaatactgtctgaattcacaacactatatgtatgcgcttcacaacaatgatcttcacaagtatacaaaatactcactcgttttggggttttaattggagccagagtatcaagatcagccgccgggaattcgactcccttactcttcaagtcatcgtatgattgtacaactccggcaagtgtag

SEQ ID NO: 8 - DNA, 98 bp, Artificial
  sequence of F46H6 and C07A12 as entered in Genbank
atgttaatattatttcagtagaagacgatgccacctctgccaagcgcaaacttgaggagttgaagaatgaacaaggagaggtaggacagcgttgagat

SEQ ID NO: 9 - DNA, 96 bp, Artificial
  sequenced genomic DNA from wild-type animals and from pBY1015
atgttaatattatttcagtagaagacgatgccacctcgcaagcgcaaacttgaggagttgaagaatgaacaaggagaggtaggacagcgttgagat

SEQ ID NO: 10 - DNA, 60 bp, Artificial
  second exon predicted by the AceDB computer program
gaagacgatgccacctctgccaagcgcaaacttgaggagttgaagaatgaacaaggagag

SEQ ID NO: 11 - DNA, 58 bp, Artificial
  first exon together with the SL1 leader sequence
gaagacgatgccacctcgcaagcgcaaacttgaggagttgaagaatgaacaacgacag

SEQ ID NO: 12 - DNA, 55 bp, Artificial
  5′ end of the shorter cDNA
gacgatgccacctcgcaagcgcaaacttgaggagttgaagaatgaacaaggagag

SEQ ID NO: 13 - DNA, 50 bp, Artificial
  5′ end of the longer cDNA
tgccacctcgcaagcgcaaacttgaggagttgaagaatgaacaaggagag

SEQ ID NO: 14 - DNA, 11 bp, Artificial
  sequence found in Genbank containing error leading to reading frame shift
acctctgccaa

SEQ ID NO: 15 - DNA, 9 bp, Artificial
  corrected sequence of SEQ ID No 14
acctcgcaa

SEQ ID NO: 16 - protein, 23 aa, Artificial
  Zn finger 1
YKCHLCGQCFYRGCGLASHLRRH

SEQ ID NO: 17 - protein, 24 aa, Artificial
  Zn finger 2
FDCEHCAYTCKHKYAYDRHLLQSH

SEQ ID NO: 18 - protein, 23 aa, Artificial
  Zn finger 3
YKCPLCISTFGSHARAVYHILSH

SEQ ID NO: 19 - protein, 23 aa, Artificial
  Zn finger 4
YLCRNCPYVSWNVSSLWRHFRHH

SEQ ID NO: 20 - protein, 23 aa, Artificial
  Zn finger 5
WTCIACSYSSSSRVKIDLHVKMH

SEQ ID NO: 21 - protein, 23 aa, Artificial
  Zn finger 6
RECTDCPFKHNDLQQFRLHRDKH

SEQ ID NO: 22 - protein, 24 aa, Artificial
  Zn finger 7
HTCPECNYSSNNHNQVVEHTFVDH

SEQ ID NO: 23 - protein, 18 aa, Artificial
  bipartite nuclear localization sequence
RRINKNDLNKPTNKKKKP

SEQ ID NO: 24 - protein, 14 aa, Artificial
  C-terminal nuclear localization sequence
PQRKKKAPKRGKRR

(19) United States
(12) Patent Application Publication
(10) Pub. No.: US 2008/0168573 A1
(43) Pub. Date: Jul. 10, 2008

(54) IDENTIFICATION OF SES-1 AND THE USES OF THE SAME

(75) Inventors: Ralf BAUMEISTER, Freiberg (DE); Bernard LAKOWSKI, Paris (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS DEUTSCHLAND GMBH, Frankfurt am Main (DE)

(21) Appl. No.: 12/036,338

(22) Filed: Feb. 25, 2008

Related U.S. Application Data
(62) Division of application No. 10/481,582, filed on Aug. 23, 2004.

(30) Foreign Application Priority Data
Jun. 22, 2001 (DE) 10130166.9

(51) Int. Cl. A01K 67/00

(52) U.S. Cl. 800/13

(57) ABSTRACT

The invention relates to isolated nucleic acid molecules coding for SES-1 proteins or muted SES-1 proteins, and vectors and transgenic organisms containing such nucleic acid molecules. The invention also relates to uses of such nucleic acid molecules for producing pharmaceuticals and for producing model organisms. The invention further relates to the corresponding SES-1 proteins and muted SES-1 proteins, and the antibodies induced thereby. Finally, the invention relates to the use of substances which increase the expression of human presenilin, for the treatment of Alzheimer's disease, in addition to said substances themselves and pharmaceutical compositions containing the same.

The present invention relates to isolated nucleic acid molecules which encode ses-1 or mutated ses-1, to vectors and transgenic organisms which contain these nucleic acid molecules, to uses of these nucleic acid molecules for producing pharmaceuticals and for generating model organisms, to the use of transgenic organisms in methods for identifying substances which alter ses-1 activity or, respectively, increase presenilin activity (e.g. that of sel-12 or hop-1), and to the corresponding SES-1 proteins and mutated SES-1 proteins as well as antibodies to which these proteins give rise. The invention furthermore relates to the use of substances which increase the expression of a human presenilin for treating Alzheimer's disease, and to these substances themselves and to pharmaceutical compositions which comprise these substances.

### BACKGROUND OF THE INVENTION

Aside from Parkinson's disease, Alzheimer's disease is that neuro-degenerative disease which is most well known. The characteristic feature of Alzheimer's disease is the development of neuronal protein aggregations, what are termed plaques, which are essentially composed of an insoluble peptide, of 4 kDa in size, termed amyloid beta-peptide (Aβ4). Investigations carried out in the last few years indicate that the formation of Aβ4 is causatively involved in the development of the disease. Dominant mutations in three genes give rise to familial forms of the disease (FAD, familial Alzheimer's disease). It has been found that one of the genes concerned encodes amyloid precursor protein (APP), which can be processed by three proteases, resulting in the formation of Aβ4, inter alia. FAD mutations in APP increase the quantity of Aβ42, a 42 amino acid-long variant of Aβ4, which is produced.

Molecular genetic investigations of families in which Alzheimer's disease has occurred have led to the identification of the human genes presenilin 1 and presenilin 2 (Rogaev et al. 1995, Nature 376, 775-778; Levy-Lahad, E. et al. 1995, Science 269: 973-977) which, when altered by particular mutations, are causatively involved in the onset of Alzheimer's disease and which also play a key role in the Notch signal transduction pathway during development of the disease. Presenilin proteins are located in the ER-Golgi and possess at least 6 transmembrane domains. It has been found that mutations in PS1 and PS2 influence the formation of Aβ4 and are involved in giving rise to Alzheimer's disease, presumably by means of the formation of amyloid deposits. Members of the presenilin family have been identified in the mouse, in Drosophila melanogaster, in Xenopus laevis and in the threadworm Caenorhabditis elegans, inter alia. Although mutated presenilins influence the processing of APP in humans, their natural function in humans is not known in detail; it has been postulated that they might function as an APP protease (gamma-secretase). Other investigations carried out on human cell cultures have indicated that presenilins play a role in the intracellular proteolysis of Notch-like receptors after they have been activated by ligand binding. It is assumed, therefore, that the presenilins are involved in processing at least two different membrane proteins (APP and Notch). At present, it is a controversial matter as to whether the presenilins themselves are the proteases in this processing or whether they are cofactors of these reactions or influence the proteolysis indirectly (Haass, C. et al. 1999, Science 286 (5441): 916-919).

Taking the abovementioned presenilin genes as the starting point, intensive research is being carried out to elucidate the molecular disease processes and to develop pharmaceuticals for treating and preventing Alzheimer's disease. Factors which influence the activity of the presenilin genes are being sought, in particular, particular importance being attached, in this connection, to the search for suppressors of the presenilin mutant phenotype. Suppressors can be mutations in other genes which eliminate the requirement for the presenilin function or they can be substances which modulate the activity of other genes, or of the presenilins, such that the presenilin mutant defect does not have any phenotypic consequences.

A nematode, in particular Caenorhabditis elegans, has frequently been employed as a preferred model organism in these investigations. The advantage of a nematode model, in particular of the C. elegans model, lies, in particular, in its suitability for a high-throughput method (HTS; high-throughput screening), the possibility of faster genetic analysis due to a shorter generation time (2-3 days) and detailed knowledge of the molecular and functional properties of the nervous system in C. elegans. Since C. elegans can be kept on microtiter plates, it is possible to use this test system to test, by means of HTS, 10 000 or more substances on the living worm over a short period of time.

Three presenilin genes, designated sel-12, hop-1 and spe-4, have thus far been identified in the threadworm C. elegans. Spe-4 is the most divergent member of the family. Mutations in spe-4 lead to a defect in cytoplasmic partitioning during spermatogenesis (L'Hemault, S. W. et al. 1992, J Cell Biol 119: 55-68). While mutations in hop-1 do not have any visible phenotype, they lead, in combination with mutations in sel-12, to a synthetically lethal phenotype (Li, X. et al. 1997, Proc Natl Acad Sci USA 94: 12204-12209). Sel-12 is the gene which most strongly resembles human PS1 and PS2 and is also the best-studied presenilin gene in C. elegans. It has been found that certain sel-12 mutants exhibit an egg-laying defect and also morphological changes in vulva development (Levitan, D. et al. 1995, Nature 377: 35-14). Sel-12 is 50% identical with human presenilins, and the egg-laying defect in sel-12 mutants can be offset by transgenically expressing PS1 or PS2 from the sel-12 promoter (Baumeister et al., 1997, Genes and Function 1: 149-159). This demonstrates that PS1 and PS2 exhibit functional homology with sel-12. Sel-12 mutations were initially identified as suppressors of the multivulva phenotype of a gain-of-function mutant in the lin-12 and glp-1 Notch receptor gene. Notch receptors control determination of cell fate in many multicellular organisms. In addition, it has been shown that the phenotype of sel-12 mutants resembles that of weak function-loss mutants of lin-12. In summary, it can be stated that presenilins play an important role in Notch signal transduction in a large number of organisms, including mammals.

### SUMMARY OF THE INVENTION

The present inventors have now found, surprisingly, that certain mutations, as shown in FIG. 1 for example, in another gene, i.e. the C. elegans ses-1 gene, which was first identified by the inventors, are able to suppress a defect, in particular the egg-laying defect of a sel-12 mutant. This means that particular mutations in the ses-1 gene lead, for example, to the egg-laying defect, which is caused by certain sel-12 mutations, being eliminated and the double mutants (sel-12⁻; ses-1⁻) once again exhibiting a normal wild-type phenotype. More detailed investigations have shown that these mutations in the ses-1 gene lead, in the sel-12 mutants, to hop-1 being activated, i.e. the hop-1 presenilin protein takes over the function of the defective sel-12 presenilin protein.

The present inventors have located the novel ses-1 gene on the C. elegans genome and sequenced it.

Consequently, the present invention initially relates to an isolated nucleic acid molecule which encodes SES-1 protein and to other nucleic acid molecules which encode mutated SES-1 proteins.

In addition to this, the invention relates to a vector which comprises these isolated nucleic acid molecules, or fragments thereof.

The invention furthermore relates to the SES-1 proteins and mutated SES-1 proteins, or fragments thereof, which are encoded by the nucleic acid molecules and to antibodies which are generated using these proteins.

In addition, the invention relates to a transgenic, nonhuman organism which comprises an isolated nucleic acid molecule which encodes SES-1 protein or mutated SES-1 protein. In particular, the invention relates to transgenic C. elegans organisms which exhibit an ses-1 allele which decreases the activity of ses-1 and, respectively, increases the activity of sel-12 or hop-1. In this connection, transgenic organisms are understood as being those organisms in which the genome has been altered by mutation, and which exhibit a mutated ses-1 gene as a result, or from which the ses-1 gene has been removed.

Finally, the invention relates to uses of these nucleic acid molecules for producing a pharmaceutical directed against Alzheimer's disease and for generating model systems for investigating this disease still further.

In particular, the invention relates to the use of transgenic C. elegans in a method for identifying and/or characterizing substances which reduce the activity of ses-1 or, respectively, increase the activity of sel-12 or hop-1.

The invention furthermore also relates to the use of substances which increase the expression of human presenilin 1 or 2 for treating Alzheimer's disease and to these substances themselves and to pharmaceutical compositions which comprise these substances.

### DETAILED DESCRIPTION OF THE INVENTION

The present invention is based on the fact that it has been possible to identify a novel gene, ses-1, in C. elegans, with this gene interacting with the C. elegans presenilin genes sel-12 and hop-1 and thereby playing an important role in the development of Alzheimer's disease.

Thus, various mutations in the ses-1 gene, including complete deletion of the ses-1 gene as well, resulted in certain altered phenotypes which were elicited by sel-12 mutations being suppressed. The suppression of these sel-12 defects in ses-1 mutants is achieved by the latter markedly derepressing-expression of hop-1 and by the HOP-1 protein presumably being able to take over the function of the defective sel-12 protein.

The ses-1 gene which has been identified contained up to 7 predicted C2H2 zinc finger domains and two regions which could serve as nuclear localization signals (FIG. 4).

When the BLAST program was used to compare its sequence with the Genbank database, the deduced SES-1 protein did not show any clear similarity to any other known protein. All that was observed was a slight similarity to a variety of proteins which also contains zinc finger domains.

Consequently, the present invention provides an isolated nucleic acid molecule which encodes SES-1 protein.

The invention furthermore encompasses isolated nucleic acid molecules which encode mutated SES-1 proteins. In particular, the invention provides isolated nucleic acid molecules which encode mutated SES-1 proteins which have at least one of the mutations given in FIG. 1.

In addition to this, other aspects of the invention relate to isolated nucleic acid molecules, in particular DNA, such as cDNA or genomic DNA, molecules, but also RNA molecules, for example, which encode a C. elegans SES-1 protein having the amino acid sequence given in SEQ ID NO: 1 or SEQ ID NO: 2, in particular those which exhibit a nucleic acid sequence given in SEQ ID NO: 3, SEQ ID NO: 4, SEQ ID NO: 5, or SEQ ID NO: 6.

The invention also encompasses isolated nucleic acid molecules whose nucleotide sequences exhibit at least 75%, in particular at least 80%, especially at least 85%, preferably at least 90%, particularly preferably at least 95% and most preferably at least 98%, sequence similarity to the abovementioned nucleic acid molecules, in particular those which hybridize with the abovementioned nucleic acid molecules under stringent conditions. Such a hybridization preferably takes place under conditions of low stringency; in another embodiment, it also takes place under conditions of high stringency. In the context of this description, conditions of low stringency are understood as meaning a hybridization in 3×SSC at from room temperature to 65° C. and conditions of high stringency are understood as meaning a hybridization in 0.1×SSC at. 68° C. SSC is the abbreviation for a 0.15 M sodium chloride, 0.015 M trisodium citrate buffer.

The nucleic acid molecules according to the invention having nucleic acid sequence similarity to the nucleic acid sequence given in SEQ ID NO: 3, SEQ ID NO: 4, SEQ ID NO: 5, or SEQ ID NO: 6 also comprise, in particular, allelic variants of the given nucleic acid sequence, which variants include, in particular, the abovementioned mutations.

The invention also extends to the nucleic acid molecules which in each case have a nucleic acid sequence which is complementary to the nucleic acid sequences described above.

In the present context, the term “isolated nucleic acid molecule” refers to nucleic acid molecules which are present in a form in which they are essentially purified from the main quantity of nucleic acid molecules of differing nature derived from the starting cells or producing cells. However, preparations of isolated nucleic acid molecules according to the invention can perfectly well contain other constituents such as salts, substances from the medium or residual constituents of the producing cells, such as various proteins.

The invention also encompasses vectors which contain a nucleic acid molecule according to the invention which encodes SES-1 protein or mutated SES-1 proteins. The invention also includes vectors which contain a fragment of a nucleic acid molecule according to the invention. In this connection, the nucleic acid molecule is linked to a promoter which causes the nucleic acid molecule to be expressed in the organism which is used for the expression. In this connection, one of the known C. elegans promoters (for example, information may be found on the world wide web at “chinook.uoregon.edu”) is used, in particular, for expressing in C. elegans.

The vector can, for example, be a plasmid, a cosmid, a bacmid, a virus or a bacteriophage.

Another aspect relates to the polypeptides or proteins which are encoded by the described nucleic acid molecules, in particular C. elegans SES-1 protein having the amino acid sequence given in SEQ ID NO: 1 or SEQ ID NO: 2 and the abovementioned mutated SES-1 proteins and also polypeptides or proteins which are derived therefrom by means of substitution, modification, deletion, insertion or addition of amino acids and which exhibit at least 75%, in particular at least 80%, especially at least 85%, preferably at least 90%, particularly preferably at least 95% and most preferably at least 98% sequence similarity to the abovementioned amino acid sequences. The invention also encompasses fusion proteins which contain the polypeptides, proteins or protein fragments according to the invention fused to a protein of a different nature or to a protein segment of a different nature, e.g. a marker protein or indicator protein. The invention also includes fragments of the abovementioned proteins or polypeptides.

The antibodies which can be obtained using these proteins, and which can be monoclonal or polyclonal, are likewise encompassed by the invention.

The invention also extends to transgenic organisms, in particular microorganisms, e.g. bacteria, viruses, protozoa, fungi and yeasts, algae, plants or animals, and also parts, e.g. cells, and propagation materials, e.g. seed, from these transgenic organisms, which comprise a recombinant nucleic acid sequence, where appropriate integrated into a chromosome or else extrachromosomally, which contains, as a transgene, a nucleic acid molecule according to the invention which encodes a SES-1 protein or mutated SES-1 protein, or fragments thereof. Under a preferred aspect, the transgenic organisms will also express the polypeptide or protein encoded by the abovementioned transgene, i.e. C. elegans SES-1 protein or a polypeptide or protein which is derived therefrom.

According to one aspect of the invention, transgenic C. elegans organisms exhibit an ses-1 allele which decreases, amplifies or eliminates the activity of ses-1. The invention also includes those transgenic C. elegans organisms from which the ses-1 gene has been completely removed. In particular, the present invention relates to transgenic C. elegans organisms which exhibit an ses-1 allele which offsets the egg-laying defect phenotype (Egl) in sel-12 mutants. In addition, the present invention relates to transgenic C. elegans organisms which exhibit an ses-1 allele which increases the activity of hop-1 or sel-12.

The invention also encompasses the use of the nucleic acid molecule according to the invention for producing a pharmaceutical for treating neurological, in particular neurodegenerative, diseases, particularly preferably Alzheimer's disease, or for generating a model organism for the further investigation and elucidation of neurological, in particular neurodegenerative, diseases.

The abovementioned pharmaceuticals can be obtained, for example, by employing a nucleic acid molecule according to the invention itself as a gene therapy agent or by using such a nucleic acid molecule to construct a model organism and formulating the substances which are found using this model organism into a pharmaceutical. For further details in this regard, the reader is referred, for example, to international application PCT/EP01/03214 belonging to the same applicant.

In particular, the invention relates to the use of transgenic C. elegans organisms, which contain a nucleic acid molecule according to the invention, in a method for identifying and/or characterizing substances which alter, in particular decrease or eliminate, the activity of ses-1. The invention furthermore relates to the use of transgenic C. elegans organisms, which contain a nucleic acid molecule according to the invention, in a method for identifying and/or characterizing substances which increase the activity of hop-1 or sel-12.

The invention furthermore relates to the use of these same transgenic C. elegans organisms in a method for identifying and/or characterizing substances which can be used as active compounds for treating and/or preventing Alzheimer's disease. In view of the analogy with human cells, and against the background of the demonstrated functional similarity of the Notch signal pathway and presenilin function in C. elegans and humans, it is assumed that the malfunction of the FAD presenilin mutants can be offset in an equivalent manner by mutating a gene which is homologous, or functionally equivalent, to ses-1 in humans. Within the context of the invention, it is expected, therefore, that a defect in a human ses-1 homolog, or a gene which is functionally equivalent, will lead to an increase in the expression of the presenilin gene which is not affected by the FAD mutation.

The invention therefore also relates to the use of substances which increase the expression of a human presenilin for treating Alzheimer's disease, and to these substances themselves. In particular, the substances according to the invention include those which activate human presenilin gene 1 or presenilin gene 2 and increase the expression of human presenilin protein 1 or human presenilin protein 2. The substances according to the invention also include those which, in C. elegans, lead to a change in ses-1 activity and/or increase sel-12 or hop-1 presenilin activity. The invention also encompasses pharmaceutical compositions which comprise these substances.

In an analogous manner, this invention can also be of benefit in the treatment of the sporadic cases of Alzheimer's disease which are age-dependent and not coupled to mutations. In this connection, it is expected that the decrease in expression will, by increasing presenilin activity, decrease the formation of Aβ4, in particular of Aβ42, and thereby chronologically delay the onset of the disease.

### BRIEF DESCRIPTION OF THE FIGURES AND OF THE SEQUENCE LISTING

FIG. 1: Ses-1 mutations.

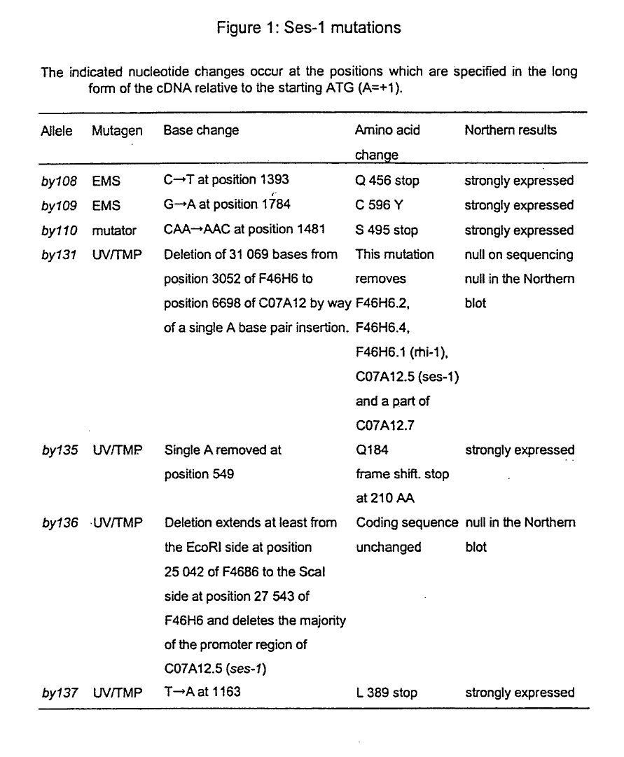

FIG. 2: Northern blot, hybridized with a hop-1-specific probe

FIG. 3: 5′ end of the ses-1 mRNAs which were found: SEQ ID NO: 8, sequence of F46H6 and C07A12 as entered in Genbank; SEQ ID NO: 9, sequenced genomic DNA from wild-type animals and from pBY1015, a clone which complements ses-1 mutants (subclones of the F36H6 cosmid); SEQ ID NO: 10, second exon predicted by the AceDB computer program; SEQ ID NO: 11, first exon, together with the SL1 leader sequence, as found by means of RT-PCR; SEQ ID NO: 12, 5′ end of the shorter cDNA; SEQ ID NO: 13, 5′ end of the longer cDNA; SEQ ID NO: 14, sequence which was found on Genbank containing a sequence error which would lead to a reading frame shift; SEQ ID NO: 15, corrected sequence of SEQ ID NO: 14.

FIG. 4: Domain structure prediction for SES-1 protein: SEQ ID NO: 16, Zn finger 1; SEQ ID NO: 17, zn finger 2; SEQ ID NO: 18, Zn finger 3; SEQ ID NO: 19, Zn finger 4; SEQ ID NO: 20, Zn finger 5; SEQ ID NO: 21, Zn finger 6; SEQ ID NO: 22, Zn finger 7; SEQ ID NO: 23, bipartite nuclear localization sequence; SEQ ID NO: 24, C-terminal nuclear localization sequence.

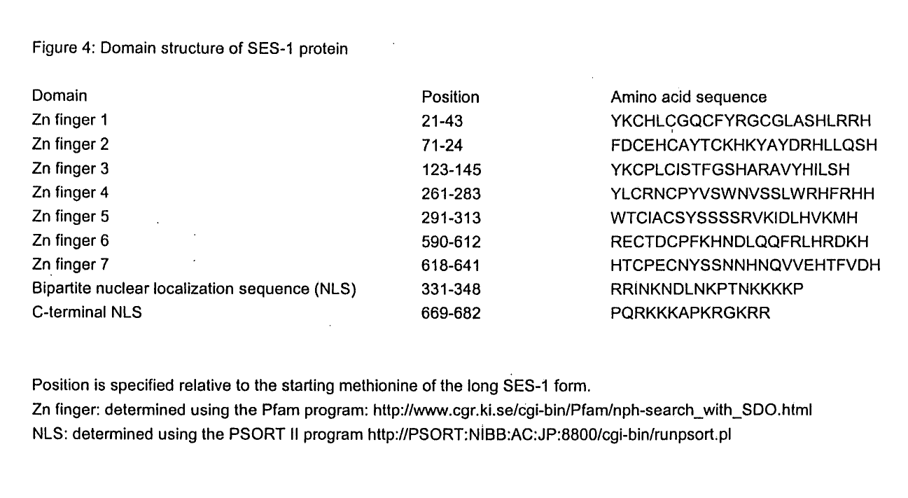

SEQ ID NO: 1: SES-1 amino acid sequence, long form.

SEQ ID NO: 2: SES-1 amino acid sequence, short form.

SEQ ID NO: 3: cDNA which encodes the long form of the SES-1 protein, from ATG to stop (yk64e9).

SEQ ID NO: 4: cDNA which encodes the short form of the SES-1 protein, from ATG to stop (yk247e5).

SEQ ID NO: 5: cDNA which encodes the long form of the SES-1 protein, from ATG to stop (yk64e9)+noncoding 3′ end region.

SEQ ID NO: 6: cDNA which encodes the long form of the SES-1 protein, from ATG to stop (yk64e9)+noncoding 3′ end region.

SEQ ID NO: 7: Sequence of pBy1015; this segment complements the defect of the ses-1 null mutant.

The invention is explained in more detail below with the aid of examples.

### EXPERIMENTAL SECTION

Isolating ses-1 Mutants

EMS and UV/TMP mutagenesis were carried out in accordance with published methods (elegans.swmed.edu). In order to identify sel-12 suppressors, L4 stage sel-12 mutants were exposed to the mutagen (e.g. ethyl methanesulfonate) and, after a reconvalescence period, were plated out in groups of 4 or 5 on 9 cm Petri dishes. In all the tests, the egg laying of the animals was analyzed in the F2 stage after the mutagenesis. Sel-12 null mutants did not normally lay any eggs. The F3 generation of the isolated animals was tested once again and those animals which laid at least 5 eggs per adult animal in both generations were isolated. In this way, homozygous suppressor strains were generated. All the mutations were backcrossed several times with wild-type animals in order to remove background mutation. Powerful suppressor mutations led to the egg-laying behavior of the animals being comparable to that of the wild-type animals despite the sel-12 null mutant being homozygous. One such suppressor mutation was termed an ses-1 mutation. Typically, ses-1/ses-12 double mutants laid approx. 250-350 eggs per animal.

The ses-1 Phenotype

Mutations in ses-1 suppress the egg-laying defect of sel-12 mutants completely. Under the stereomicroscope, it is possible to distinguish 3 aspects of the sel-12 egg-laying defect. About 75% of all the sel-12(ar171) animals exhibit a marked bulging of the vulva, which is known as the protruding vulva phenotype (Pvl). The precise reason for this defect has not been reported. Virtually all the sel-12(ar171) animals contain more eggs in the uterus than do comparable wild-type animals, whose constant egg number is approx. 15 whereas this number is up to 60 eggs in the case of sel-12. This leads to the animals becoming distended, i.e. what is described as the Egl phenotype. The eggs accumulate in the uterus and the embryos hatch within the uterus and continue to develop in the mother, which in virtually 100% of all cases dies prematurely in connection with this. The phenotype is termed the “bag of worms” phenotype. Wild-type animals are observed extremely rarely in homozygous sel-12 culture. Ses-1 mutations suppress all three aspects of this sel-12(ar171) phenotype virtually completely. Sel-12(ar171); ses-1 animals are slightly smaller than wild-type animals and contain fewer eggs in the uterus than normal (a weak Egl-constitutive phenotype). This suggests that egg laying in the double mutant is slightly overstimulated. Ses-1 allelic animals also suppress the egg-laying defects of all the other sel-12 mutations which have been described. This proves that ses-1 mutations do not act in an-allele-specific manner.

Cloning the ses-1 Gene Locus

Ses-1 was mapped on the left arm of chromosome X. For this, sel-12(ar171) ses-1 strains were positioned using a genetic marker mutation, egg-laying-defective (Egl) recombinant animals were isolated and a test was carried out to determine how many of these animals had received the marker mutation. In order to map ses-1 more closely, the presence/absence of ses-1 mutations in a strain was measured by suppression of the sel-12(ar171) egg-laying defect. For this, sel-12(ar171) ses-1 males were crossed with AB double-mutant strains, with A and B being two mutations which are located to the right of sel-12 on the genetic map and with B being the right-hand of the two markers. Recombinants which were only B recombinants, and which did not carry the A mutation, were then sought. As a result of this recombination method, all the recombinants should contain sel-12(ar171) apart from those which carried a double recombination. By means of a series of crossing experiments, ses-1 was mapped between dpy-23(e840) and lin-2(e678), approx. ⅕th of the distance from dpy-23(e840) to lon-2. All the experiments indicated that ses-1 mutations bring about a loss of function. In order to demonstrate this, ses-1 mutations were positioned by way of two free duplications. Although the ses-1 alleles by108 and by109 mutations do not complement each other, the possibility existed that both represent mutations in two different genes on the left arm of chromosome X. For this reason, by108 and by109 were initially mapped independently of each other. In order to verify the mappings, two duplications, i.e.; mnDp31 and mnDp32, were transferred into the strain sel-12(ar171) ses-1(by108) lon-2(e678) and tested for egg laying.

The strain sel-12(ar171) ses-1(by108) lon-2(e678); nDp31, which carries two mutated copies of the ses-1 gene on the X chromosome, and also possesses a wild-type copy on an extrachromosomal array DNA, possesses an entirely ses-1 wild-type phenotype, which proves that the tested mutants are function-loss mutants. By linearly interpolating the data, ses-1 was located approx. 50 kb to the left of dpy-23(e840) and the transgenic suppression was subsequently tested using cosmids of this chromosome region.

Transgene Rescue of the ses-1 Defect

Transgenes were injected into the strain sel-12(ar171) ses-1(by108) and the antisuppressor activity of the transfected clones was tested. All the clones were injected at a concentration of 20 ng/μl together with 100 ng of pRF4 μl and 20 ng of pBY218/μl as transformation markers. The ses-1 phenotype was completely offset (rescued) by injecting the cosmid F46H6. After that, F46H6 subclones, purified restriction fragments and PCR products from this cosmid were tested in order to elucidate the identity of the ses-1 gene.

Isolating F46H6 Subfragments

F46H6 was cut with various restriction enzymes. Subfragments were purified using Qiaquick gel extraction kits. A 21.5 kb Ncol fragment and a 0.2 kb BamHI fragment (in the plasmid pBY1015) were injected directly and (both) rescue the phenotype. The minimum rescuing fragment was reduced to a sequence of 4 kb. This segment contains only one gene, which encodes a protein possessing a large number of zinc fingers.

Structure of ses-1

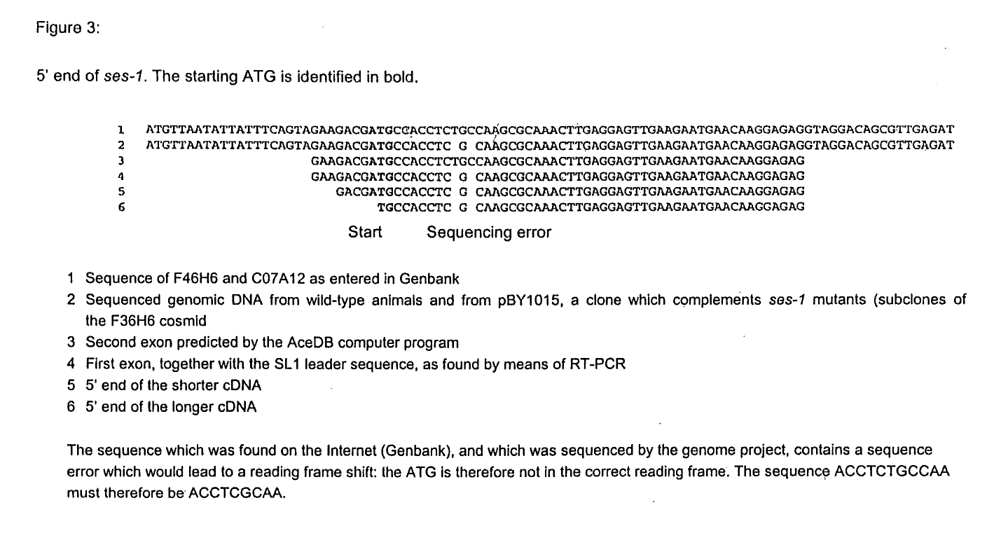

Two cDNAs of ses-1 were identified and contained 1.9 kb and 2.1 kb of sequence. The two cDNAs contain identical DNA sequences and exon-intron boundaries, with one exception: the larger clone carries an additional intron while the small clone has a 5′ end which is longer by 6 bases (in this regard, see FIG. 3).

The sequenced cDNAs differ at several locations from the gene predicted in the databases. In the first place, the 5′ end which was found is markedly shorter than predicted by computer analysis. In the second place, sequence differences were found between both cDNAs and the genomic sequences, published on the Internet, of the region on cosmids F46H6 and C07A12 (neighbor cosmid) (FIG. 3).

In addition, ses-1 is trans-spliced to an SL1 leader sequence. The SL1-specific product was sequenced. It was found that the leader sequence is connected immediately 5′ to the two predicted 5′ ends of the cDNAs.

In Northern blot experiments, a band which can be detected in all the C. elegans development stages was only found in the case of the longer cDNA. The shorter cDNA could be an artifact; alternatively, it might only appear in small, undetectable quantities but could nevertheless perfectly well exhibit biological activity.

Intracellular Location

A ses-1 fusion gene was expressed in a bacculovirus SF9 cell system and the location was identified by immunostaining. Ses-1 is located in the nucleus; this is in agreement with its predicted role as a transcription factor.

Expression

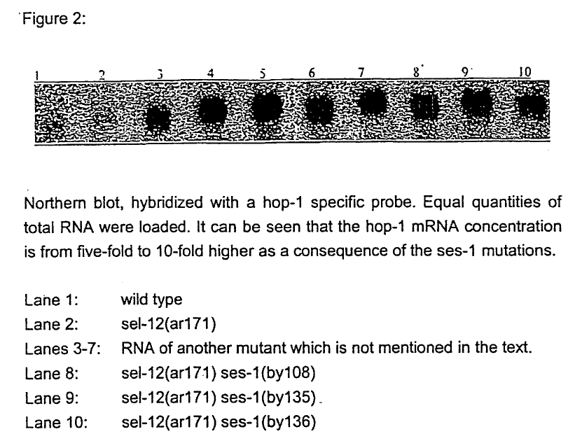

The Northern blot analysis showed that ses-1 transcripts are expressed in all development stages (FIG. 2).

The ses-1 Null Phenotype

By131 is a mutant which can no longer have any ses-1 function (deletion of the entire gene). However, this deletion also eliminates neighboring genes. By135 leads to a reading frame shift, as described, and should therefore be the most powerful ses-1 mutant. Ses-1(by135) and by131 animals were investigated. Both strains of phenotypically wild-type, both morphologically and on the basis of their egg-laying behavior. The progeny numbers of the two animals are somewhat lower than in the wild type. Both alleles completely suppress the sel-12 phenotype of all alleles and also lead to the sel-12 alleles having wild-type progeny number. It can therefore be concluded that ses-1 is a specific suppressor of sel-12.

All the defects of sel-12 mutants, or of sel-12, hop-1 double mutants, can be attributed to effects of these presenilins in the lin-12 and glp-1 Notch signaling pathway.

Since ses-1 is a nuclearly expressed gene, which could act as a transcription factor, an investigation was carried out to determine whether the ses-1 mutations are able to alter the transcription of other genes of the lin-12/glp-1 Notch signaling pathway. Stage-specific RNAs were isolated from the wild type and various ses-1 mutant strains and analyzed in Northern blots. The samples were tested by hybridizing with hop-1 cDNAs. Hop-1 is strongly expressed in the eggs of wild-type animals but is almost impossible to detect in the L1 larval stage. Expression increases slowly from the L2 stage to the adult stage and reaches its peak in adult animals. In sel-12(ar171) animals, the expression of hop-1 was to a large extent identical. In sel-12(ar171) ses-1(by1135) animals and ses-1(by135) animals, expression differed significantly in one stage: it was clearly possible to detect hop-1 in the L1 stage. The expression of hop-1 in the L1 stage was also significantly greater in ses-1 alleles by108 and by136. This means that ses-1 mutants achieve suppression of sel-12 defects by markedly derepressing the expression of hop-1, and HOP-1 protein is presumably able to assume the function of the defective SEL-12.

1. A transgenic C. elegans comprising:
(a) mutated sel-12 wherein the sel-12 mutation results in a sel-12 phenotype selected from the group consisting of Pvl phenotype, Egl phenotype, and “bag of worms” phenotype, and
(b) mutated ses-1 wherein the ses-1 mutation expresses a mutated form of SES-1 protein of SEQ ID NO:1 or SEQ ID NO:2 and wherein the ses-1 mutation suppresses the sel-12 phenotype.

2. The transgenic C. elegans as described in claim 2, wherein mutated ses-1 increases the activity of hop-1.
